(19) United States
(12) Plant Patent Application Publication
(10) Pub. No.: US 2019/0246544 P1
(43) Pub. Date: Aug. 8, 2019

(54) Garlic plant denominated Mary Miller

(50) ;

(71) Applicant: Allyson Jayne Flagg-Miller, Corvallis, OR (US)

(72) Inventor: Allyson Jayne Flagg-Miller, Corvallis, OR (US)

(21) Appl. No.: 15/932,116

(22) Filed: Feb. 6, 2018

(51) Int. Cl. A01H 6/04

(52) U.S. Cl. PLT/258

(57) ABSTRACT

A rediscovered historically significant Garlic plant named “Mary Miller Garlic.” Characterized by larger bulbs, yields 5 large spicy hot bulbs per plant, 7.5 to 8 foot in overall plant height, 12 to 18 month shelf life, 9 month from planting to harvest of the plant, high yield of bulbs, disease-free vegetation, presence very large spicy hot edible flower scape, and vigorous foliage that develops unusually larger bulbs and scapes.

Latin name and variety denomination:

The present disclosure relates to a new and distinct variety of Allium sativum L, which is hereby denominated ‘MARY MILLER’.

### BACKGROUND

The present invention relates to a new and distinct cultivar of garlic plant, botanically known as and hereinafter referred to by the cultivar name ‘MARY MILLER’.

‘MARY MILLER’ is a product of a planned breeding program which had the objective of creating new garlic cultivars having early harvesting capabilities, high yield potential, large clove arrangements, healthy plant material and seed-propagated garlic cultivation and has been asexually reproduced. The clones so produced have been observed through several growing seasons and the clones are identical to the parent plant in all characteristics.

Characterized by larger bulbs, yields 5 large spicy hot bulbs per plant, 7.5 to 8 foot in overall plant height, 12 to 18 month shelf life, 9 month from planting to harvest of the plant, high yield of bulbs, disease-free vegetation, presence very large spicy hot edible flower scape, and vigorous foliage that develops unusually larger bulbs and scapes.

The following traits have been repeatedly observed and are determined to be basic characteristics of ‘Mary Miller Garlic’ which in combination distinguish this garlic as a new and distinct cultivar:

1 1. 9 month harvesting of the plant;
1 2. High yield of bulbs;
1 3. Disease-free vegetation;
1 4. Presence of large, hot, edible or ornamental flower scape; and
1 5. Vigorous foliage that develops quicker and has more volume than comparative cultivars.
1.6 Cloves are hot and spicy to the taste and are medicinal.

‘Mary Miller Garlic’ has not been observed under all possible environmental conditions. The phenotype of the new cultivar may vary significantly with variations in environment such as day length, temperature, soil condition, humidity, fertilization and date of planting without any change in genotype of the cultivar. The following observations, measurements, and comparisons describe plants grown in Grape Lane Poultry Farm, in the Willamette Valley, Oreg. under conditions that approximate those generally used in commercial practice. The age of the plants described is 9 months after planting.

Of the many commercial cultivars known to the present inventor, the most similar in comparison to ‘Mary Miller Garlic’ is ‘Elephant Garlic’ (never patented). In comparison to ‘Elephant Garlic’, ‘Mary Miller Garlic ’ has similar bulb and clove colors but demonstrates significant differences in the earliness of harvesting, larger bulbs, larger scape, both scape and bulbs are very hot to the taste, longer shelf life, higher yield and healthier cultivation and culinary values.

### BRIEF DESCRIPTION OF THE DRAWINGS

The present variety will become more fully understood from the detailed description and the accompanying drawings, wherein:

FIG. 1 is a photographic print in full color of a first perspective of a ‘Mary Miller’ Garlic, wherein the plant is in the foreground and the additional plants or portions thereof in the background and the grass on the ground are not part of the ‘Mary Miller’ Garlic;

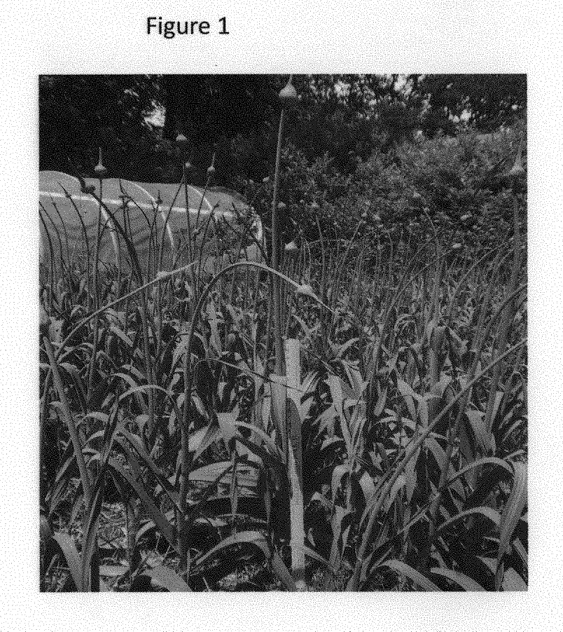

FIG. 2 is a photographic print in full color of a second perspective of a ‘Mary Miller’ Garlic, wherein the plant has been harvested;

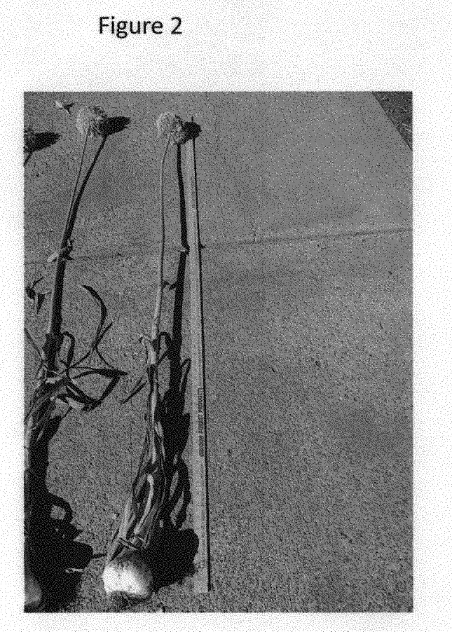

FIG. 3 is a photographic print in full color illustrating a first ‘Mary Miller’ sectional view of bulb clusters, wherein the bulbs shown are mature;

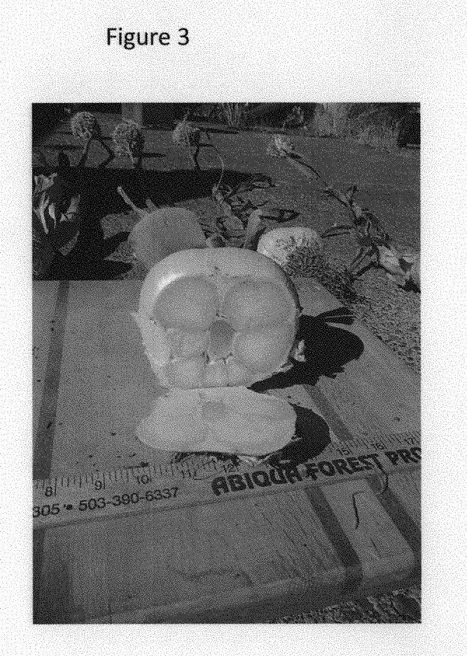

FIG. 4 is a photographic print in full color illustrating a second ‘Mary Miller’ bulb with exemplary bulb clusters; wherein most, but not all, of the bulb shown are mature; and

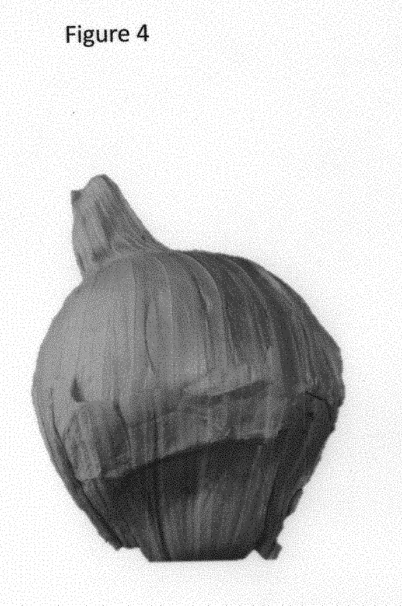

FIG. 5 is a photographic print in full color illustrating a second ‘Mary Miller’ Umbule; and

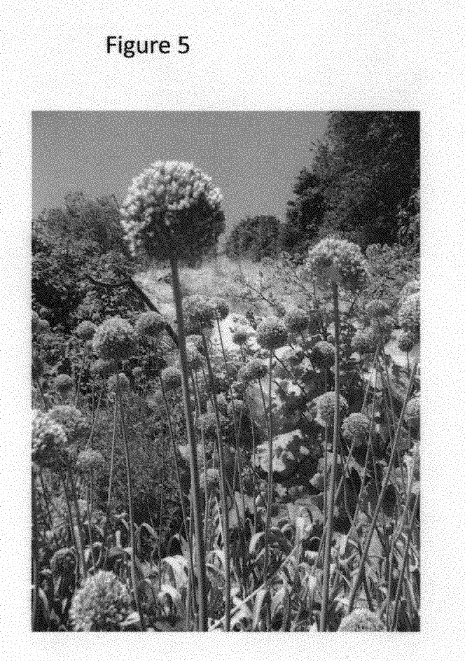

FIG. 6. diameter hard shelled pod seed.

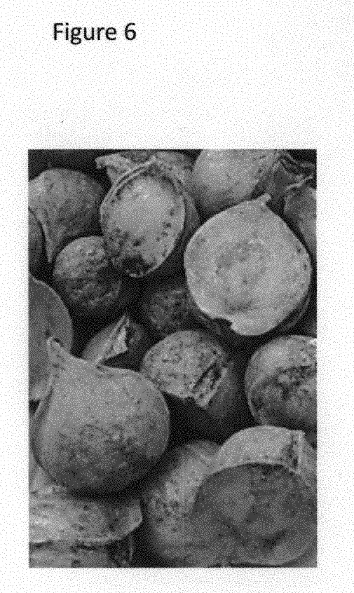

### DETAILED BOTANICAL DESCRIPTION

The following observations, measurements and values describe the rediscovered cultivar as grown at Grape Lane Poultry Farm in the Willamette Valley, Oreg., under conditions which closely approximate those generally used in commercial practice. This garlic was found and saved by Allyson Jayne Flagg-Miller.
Origin: Antique Europe, discovered at Grape Lane Poultry Farm, Willamette Valley, Oreg. by Allyson Jayne Flagg-Miller in 2000.
Parentage: Unnamed seedlings of European origin.
Classification: Botanical. — Allium A J ampeloprasum Flagg.
   Commercial.—Garlic, c.v. ‘Mary Miller Garlic’.
Plant: Garlic.
   Form.—Bulbous herb.
   Height including umbel.—6.5 to 7 feet, depending on cultivation conditions. when grown in Grape Lane Poultry Farm, Willamette Valley, Oreg.
   Growth habit.—Fast response, very vigorous growth and volume (in comparison to standard variety ‘Elephant Garlic’.
   Harvest/typical growing season.—In one growing season only one harvest takes place. The typical growing season is from October to July in Western United States climate conditions and from October to June in sub-Mediterranean conditions.
Foliage: Quantity. — 7 to 11 leaves depending on cultivation conditions.
   Size.—Minimum leaf length is 3 feet, maximum leaf length is 5 feet, depending on cultivation conditions.
   Shape of leaf.—Elongated.
   Aspect.—Arched to hanging.
   Color.—Light emerald green for both upper and lower leaf surfaces.
   Leaf width.—Small tapper from plant stem broadening to a wider center of 4 to 6 inches before tapping to a pointed tip.
   Apex.—Acute.
   Base.—Sessile.
   Texture.—Satin.
   Stem coloration.—Converts from emerald green to light green.
Scape:
   Length.—9″ to 12″ depending on cultivation conditions.
   Diameter.—12″ to 20″.
   Color.—Purple flower.
   Umbels.—Large umbels that shed their spathes at maturity.
   Bulbils.—Rare to find and are not common to this plant. I have found a total of 3 plants producing bulbils.
   Shape.—Elliptical.
   Color.—Light Brown.
   Spathe.—Sheds at maturity of plant.
   Bulb shape.—Spherical.
   Size.—Average of 17″ diameter, depending on cultivation conditions.
   Color.—Cream color to light golden.
   Number of cloves.—5 to 6.
   Clove color.—White.
   Clove skin color.—Golden yellow.
   Clove shape.—Transversely in triangular shape with round edges.
   Clove length.—4 to 6 inches longitudinal section running to a point.
   Clove diameter.—8 to 8.5 inches at the widest part.
   Clove texture.—Glossy with and without the skin.
Disease resistance: Very high for mildew, rust and botrytis. Showed stable growth and health through climate changes in weather on the Willamette Valley floor. Wetter conditions than normal.
Other characteristics: Storability over a year.
Pollen: Is produced.
Seed: A 1″ diameter hard shelled pod seed pictured below is produced yielding a garlic in 2 years.

What is claimed is:

1. A new and distinct cultivar of garlic plant named ‘Mary Miller’, as illustrated and described.
